(19) United States
(12) Patent Application Publication
(10) Pub. No.: US 2010/0100011 A1
(43) Pub. Date: Apr. 22, 2010

(54) System and Method for Orthopedic Alignment and Measurement

(76) Inventor: Martin Roche, Fort Lauderdale, FL (US)

Correspondence Address: Orthosensor, Inc., 1560 Sawgrass Corporate Pkwy, 4th Floor, Sunrise, FL, 33323, US

(21) Appl. No.: 12/604,072

(22) Filed: Oct. 22, 2009

Related U.S. Application Data
(60) Provisional application No. 61/196,914, filed on Oct. 22, 2008.
(60) Provisional application No. 61/196,915, filed on Oct. 22, 2008.
(60) Provisional application No. 61/196,916, filed on Oct. 22, 2008.

(51) Int. Cl. A61B 5/103; A61F 2/38; A61F 2/44; A61F 2/32

(52) U.S. Cl. 600/587; 623/20.14; 623/17.11; 623/22.15

(57) ABSTRACT

At least one embodiment is directed to a system for measuring parameters of a skeletal system in positions of optimal alignment for implantation of an orthopedic device. The system comprises one or more position sensors (202, 204, 206, 208, 210, and 212), one or more measurement sensors (606), a processing unit (506), and a screen (502). The position and measurement sensors are in communication with the processing unit. Position and relational positioning information in conjunction with one or more parameter measurements is used to determine proper seating of an implant, device balance over a range of motion, and device stability. For example, measurement of loading over a range of motion can be used to determine the amount and type of adjustment required for an implant. The positional and measurement data is stored in a database and accessible to the processing unit (506) to aid the surgeon, hospital, and implant manufacturer.

### CROSS-REFERENCE

This application claims the priority benefits of U.S. Provisional Patent Application No. 61/196,914, U.S. Provisional Patent Application No. 61/196,915, and U.S. Provisional Patent Application No. 61/196,916 all filed on Oct. 22, 2008, the entire contents of which are hereby incorporated by reference.

### FIELD

The invention relates in general to orthopedics, and particularly though not exclusively, is related to a device and method to implant an orthopedic joint.

### BACKGROUND

The skeletal system is a balanced support framework subject to variation and degradation. Changes in the skeletal system can occur due to environmental factors, degeneration, and aging. An orthopedic joint of the skeletal system typically comprises two or more bones that move in relation to one another. Movement is enabled by muscle tissue and tendons attached to the skeletal system of the joint. Ligaments hold and stabilize the one or more joint bones positionally. Cartilage is a wear surface that prevents bone-to-bone contact, distributes load, and lowers friction.

There has been substantial growth in the repairing of the human skeletal system as orthopedic joint implant technology has evolved. In general, improvements to orthopedic joints have been based on empirical data that is sporadically gathered. Similarly, the majority of implant surgeries are being performed with tools that have not changed substantially in decades but have been refined over time. In general, the orthopedic implant procedure has been standardized to meet the needs of the general population. Adjustments due to individual skeletal variations rely on the skill of the surgeon to adjust the process for the exact circumstance. At issue is that there is little or no data during an orthopedic surgery, post-operatively, and long term that provides feedback to the orthopedic manufacturers and surgeons about the implant status.

### BRIEF DESCRIPTION OF THE DRAWINGS

Exemplary embodiments of present invention will become more fully understood from the detailed description and the accompanying drawings, wherein:

FIG. 1 is an illustration of a mechanical axis of a lower leg in accordance with an exemplary embodiment;

FIG. 2 is an illustration of a plurality of sensor arrays placed on a lower leg in accordance with an exemplary embodiment;

FIG. 3 is a lateral view illustrating the plurality of sensor arrays placed on a lower leg in accordance with an exemplary embodiment;

FIG. 4 is a lateral view illustrating the lower leg with the plurality of sensor arrays in extension and flexion in accordance with an exemplary embodiment;

FIG. 5 is a lateral view of the plurality of sensor arrays in communication with a processor and screen for providing information in accordance with an exemplary embodiment;

FIG. 6 is a lateral view of the knee illustrating a knee with a joint implant and sensors in accordance with an exemplary embodiment;

FIG. 7 is an anteroposterio view of a knee and sensor arrays in accordance with an exemplary embodiment;

FIG. 8 is an illustration of a system having sensor arrays in accordance with an exemplary embodiment;

FIG. 9 is an illustration of a hip implant having sensor arrays in accordance with an exemplary embodiment;

FIG. 10 is an illustration of a hip implant having load sensors in accordance with an exemplary embodiment;

FIG. 11 is an illustration of moving the hip implant to measure load and position through a range of motion in accordance with an exemplary embodiment;

FIG. 12 is an illustration of a spinal column and sensor arrays in accordance with an exemplary embodiment;

FIG. 13 is an illustration of a spinal column and sensor arrays providing positional information in accordance with an exemplary embodiment;

FIG. 14 is an illustration of vertebrae having sensor arrays in accordance with an exemplary embodiment;

FIG. 15 is an illustration of a spinal implant and cage in accordance with an exemplary embodiment; and

FIG. 16 depicts an exemplary diagrammatic representation of a machine in the form of a computer system within which a set of instructions, when executed, may cause the machine to perform any one or more of the methodologies disclosed herein.

### DETAILED DESCRIPTION

The following description of exemplary embodiment(s) is merely illustrative in nature and is in no way intended to limit the invention, its application, or uses.

Processes, techniques, apparatus, and materials as known by one of ordinary skill in the art may not be discussed in detail but are intended to be part of the enabling description where appropriate. For example specific computer code may not be listed for achieving each of the steps discussed, however one of ordinary skill would be able, without undo experimentation, to write such code given the enabling disclosure herein. Such code is intended to fall within the scope of at least one exemplary embodiment.

Additionally, the sizes of structures used in exemplary embodiments are not limited by any discussion herein (e.g., the sizes of structures can be macro (centimeter, meter, and size), micro (micro meter), nanometer size and smaller).

Notice that similar reference numerals and letters refer to similar items in the following figures, and thus once an item is defined in one figure, it may not be discussed or further defined in the following figures.

In all of the examples illustrated and discussed herein, any specific values, should be interpreted to be illustrative only and non-limiting. Thus, other examples of the exemplary embodiments could have different values.

In general, the successful implantation of an orthopedic device in a skeletal system and more specifically in a joint or spine depends on multiple factors. One factor is that the surgeon strives to implant the device to obtain adequate alignment of the extremity or spine. A second factor is proper seating of the implant for stability. A third factor is that orthopedic implants typically comprise more than one component that are aligned in relation to one another. A fourth factor is balance of loading over a range motion.

By way of a device herein contemplated the surgeon receives measured data during surgery and post operatively on the factors listed above. As one example, accurate measurements can be made during joint implant surgery to determine if an implant is optimally balanced and aligned. This can reduce operating time and surgical stress for both the surgeon and patient. The data generated by direct measurement of the implanted joint can be further processed to assess joint integrity, operation, and joint wear thereby leading to improved design and materials.

As one example load balance adjustment can be achieved by soft tissue release in response to the assessment. The surgeon or device can reduce tension on one or more ligaments to modify loading to a more optimal situation. In this scenario, the surgeon receives measured data by way of the device during surgery and post operatively on the factors listed above. Consequently, the surgical outcome is a function of the device as complemented with the surgeon's abilities but not so highly dependent alone on the surgeon's skill. The device captures the “feel” of how an implanted device should properly operate to improve precision and minimize variation; including haptic and visual cues.

The surgeon utilizes surgical tools to obtain appropriate bony cuts to the skeletal system and alignment of the implanted device to the bone. The surgical tools are often mechanical devices used to achieve gross alignment of the skeletal system prior to or during an implant surgery. In a non-limiting example, mechanical alignment aids are commonly used to align the femur, tibia, and ankle optimally. The mechanical alignment aids are not integrated, take time to deploy, and have limited accuracy.

In at least one exemplary embodiment, a single system comprising one or more sensors is used intra-operatively, to define implant positioning, achieve appropriate implant orientation, and limb alignment. In particular, the system combines the ability to provide position information and measure one or more other parameters (e.g. load, blood flow, distance, etc. . . . ) that provides quantitative data to a surgeon that allows an implant to be adjusted within predetermined values or ranges based on the measured data and a database of other similar procedures. The system is designed broadly for use on the skeletal system including but not limited to the spinal column, knee, hip, ankle, shoulder, wrist, articulating, and non-articulating structures. For example, the sensors will enable the surgeon to measure joint loading while utilizing soft tissue tensioning to adjust balance and maximize stability of an implanted joint. Similarly, measured data in conjunction with positioning can be collected before and during surgery to aid the surgeon in ensuring that, the implanted device has an equivalent geometry and range of motion.

It should be noted that very little data exists on implanted orthopedic devices. Most of the data is empirically obtained by analyzing orthopedic devices that have been used in a human subject or simulated use. Wear patterns, material issues, and failure mechanisms are studied. Although, information can be garnered through this type of study it does yield substantive data about the initial installation, post-operative use, and long term use from a measurement perspective. Just as each person is different, each device installation is different having variations in initial loading, balance, and alignment. Having measured data on each installation as well as generating post-operative and long-term measured data gives significant insight on the operation of a device under widely varying conditions. In at least one exemplary embodiment, the measured data can be collected to a database where it can be stored and analyzed. For example, once a relevant sample of the measured data is collected, it can be used to define optimal initial measured settings, geometries, and alignments for maximizing the life and usability of an implanted orthopedic device. In a non-limiting example, the system disclosed herein can be used by surgeons to measure the roughed-in implant device (or trial) and then make measurements that are used to dictate further bony cuts and alignments to fine tune the implanted device to meet the optimal settings. Furthermore, one or more sensors can be implanted to monitor the joint post-operatively and long term. The one or more sensors can monitor wear or other parameter that indicates failure or degradation of the orthopedic device. Thus, the one or more sensors can indicate a problem or suggest an optimal time to replace components of the orthopedic device such that only a minimally invasive procedure is required thereby saving cost and stress on the patient. A further benefit of the system is the use of the measured data to improve materials and orthopedic implant designs based on measured parameters such as alignment, loading, balance, wear, temperature, and position.

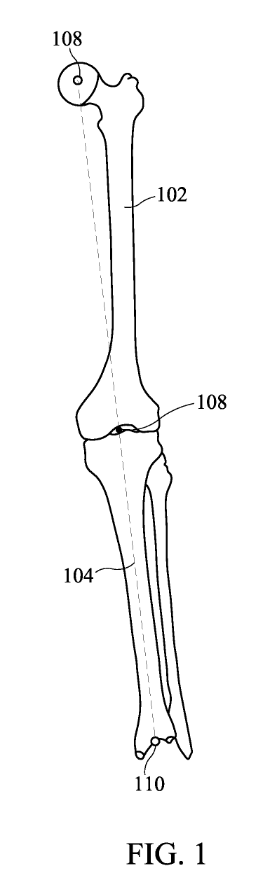

FIG. 1 is an illustration of a mechanical axis 100 of a leg in accordance with an exemplary embodiment. The lower leg comprises a femur 102 and a tibia 104. Mechanical axis 100 is typically defined with the leg in extension. The mechanical axis 100 of the lower leg corresponds to a straight line drawn from a center of the femoral head 106, through the medial tibial spine 108, and through a center of an ankle 110. In an optimal mechanical alignment, mechanical axis 100 will pass through the anatomical center of the knee in all three dimensions. This is useful as it can define an alignment in every plane of the knee.

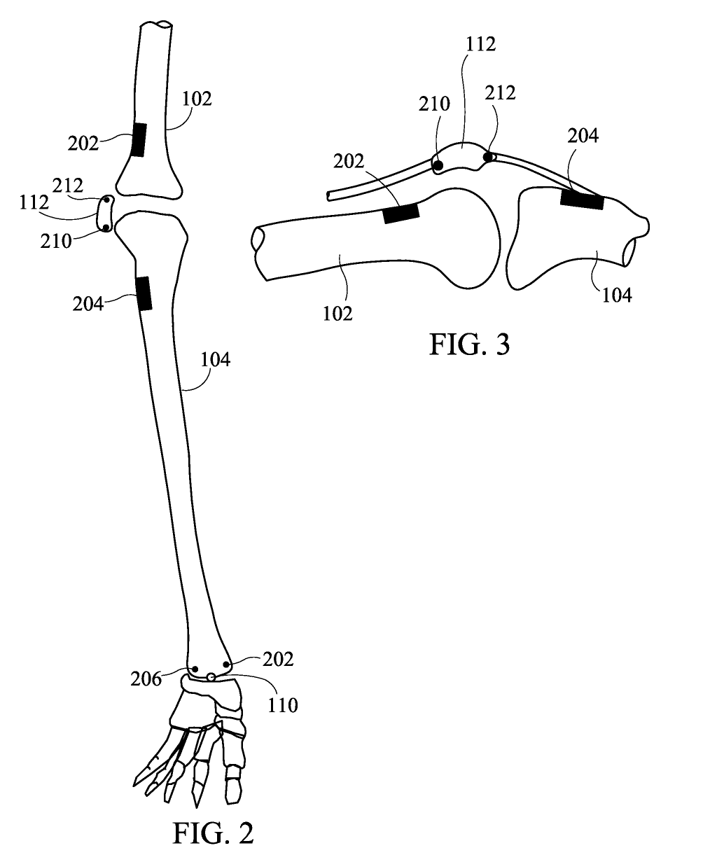

FIG. 2 is an illustration of a plurality of sensors placed on a lower leg in accordance with an exemplary embodiment. In at least one exemplary embodiment, the sensors are a component of a system that identifies position, relational positioning and measures parameters of the knee to aid in fitting of an orthopedic device. In a non-limiting example, some of sensors can be inserted in bone of the lower leg. For example, the sensors can be placed in a housing that has external screw threads. In at least one exemplary embodiment, the sensors comprise a control circuit, circuitry for wired or wireless communication, a power source (temporary or rechargeable). In a non-limiting example, a position sensor can include one or more mems accelerometers for measuring spatial orientation and position in three dimensions. A measurement sensor can include a device for measuring a parameter such as a strain gauge for measuring load or temperature sensor. The sensors in a screw type housing can then be easily attached in bone using tools common to an orthopedic surgeon. Alternatively, the sensors can be temporarily attached to the bone, an implant device, or a surgical tool so they can be removed or disposed of. The sensors can also be included in the orthopedic implant.

In a non-limiting example, the system comprises positional sensor arrays 202, 204, 206, 208, 210, and 212 attached to the skeletal system. The system measures the position of each bone in which a sensor is attached as well as the relational positioning/spatial orientation in three dimensions. In an accelerometer position sensor system, a reference position can be identified and used to determine the location of other points. Ultrasonic, infra-red, electromagnetic, and fiber optic sensors can be used as well. Sensor array 202 is coupled to femur 102. Sensor array 204 is coupled to tibia 104. Sensor array 206 and 208 are respectively coupled to the medial malleoulus and lateral malleoulus of the ankle. In at least one exemplary embodiment, sensor array 206 and 208 are formed in a sensor pad that can be attached to the ankle. The center of ankle 110 is determined from sensor arrays 206 and 208. The center of the femoral head can be determined by pre-operative scans or identified prior to alignment using a technique such as ultrasonic definition. Alternatively, one or more identification points can be registered using electro-magnetic, ultrasonic or infra-red sensors, and used in an alignment procedure to align skeletal structure. Sensor arrays 210 and 212 are coupled to a patella 112 to monitor the position of patella 112 in relation to distal end of the femur and proximal end of the tibia.

FIG. 3 is a lateral view illustrating the plurality of sensors placed on the lower leg in accordance with an exemplary embodiment. Sensor array 202 provides position information of femur 102. Sensor array 204 provides position information of tibia 104. Relational positioning information of femur 102 to tibia 104 can be indicated on a screen of the system and used in real time during orthopedic implant surgery. In general, accurate relational positioning can be used to identify a mechanical axis, initiate cuts in a predetermined position, to check that an installed device is aligned correctly, or verify a range of motion. Similarly, sensor arrays 210 and 212 can provide relational positioning information of patella 112 to femur 102 and tibia 104. Sensor arrays 210 and 212 can also include force measuring sensors to determine the loading on patella 112 such that patellar tracking and tension can be adjusted through soft tissue tensioning (and the adjustments measured and viewed). Although not shown, sensor arrays 202, 204, 206, 208, 210, and 212 are in communication with a processing unit that receives the positional and measurement information and displays the information in a format useful to a surgeon on a screen or display. It should be noted that sensors disclosed herein can be temporarily attached. In a non-limiting example, a sensor array can be taped, glued, or pinned to a location internal or external to the body. This allows additional flexibility to the placement of the sensors. The sensors can then be removed for reuse or disposed of after measurements have been taken thereby being out of the way for subsequent surgical steps if desired.

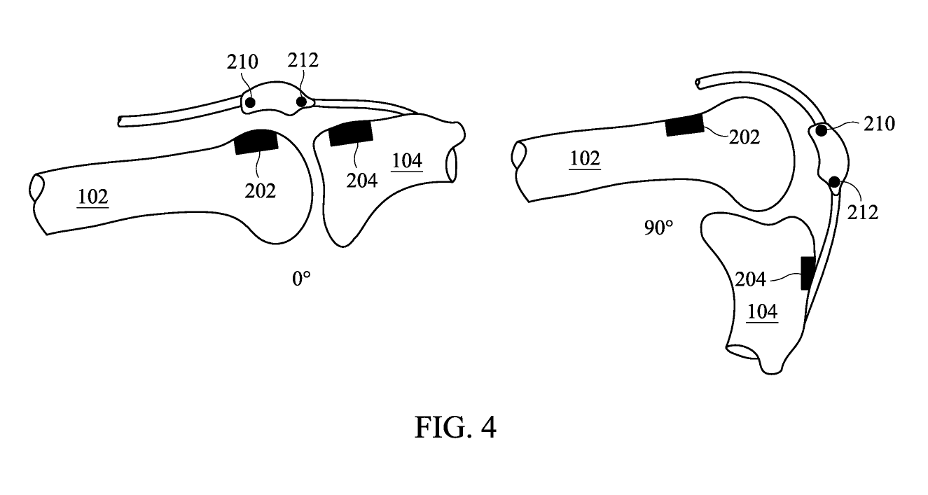

FIG. 4 is a lateral view illustrating the lower leg with a plurality of sensor arrays in extension and flexion in accordance with an exemplary embodiment. In at least one exemplary embodiment, accelerometers in sensors 202 and 204 provide positional information and relational positioning. In at least one exemplary embodiment, accelerometers are in integrated circuit form such that a small form factor can be achieved. Furthermore, accelerometers can be provided that measure all three dimensions. The accelerometers can be integrated with the control circuit to further reduce sensor array footprint.

The lower leg can be positioned in extension by the surgeon. A screen displays the relative positioning such that femur 102 and tibia 104 are positioned corresponding to an actual position of the leg. For example, the surgeon places femur 102 and tibia 104 in extension such that they are both in the same plane. The display of the system indicates the position of femur 102 in relation to tibia 104 and shows an angle (zero degrees) indicating that the leg is in extension.

A measurement of zero degrees describes femur 102 and tibia 104 in the same plane. The lower leg can be aligned to an optimal mechanical axis using position data from sensor arrays 202, 204, 206, 208, and a location of hip center 108. Alternatively, hip center 108 can be identified by rotating femur 102 and using sensor arrays 202 to track the motion. The tracked motion can be use to interpret the location of hip center 108. The knee center can be defined in the incision. Thus, the mechanical axis of the lower leg can then be defined very accurately using the sensors by aligning hip center 108, the knee center, and ankle center 110. The surgeon then has the benefit of proper alignment during the course of the implant surgery. Moreover, the positional relationship can be tracked throughout surgery. For example, in an orthopedic device implant, measurements can be taken over a range of motion to determine and ensure proper fit over the operational bounds of the device. As shown, sensor arrays 202 and 204 respectively coupled to femur 102 and tibia 104 can indicate the lower leg in flexion. More specifically, sensors 202 and 204 indicate that tibia 104 is positioned ninety degrees from a position of femur 102. Thus, the surgeon can make cuts and adjustments knowing the alignment and the positional relationships of bones of a skeletal structure are correct.

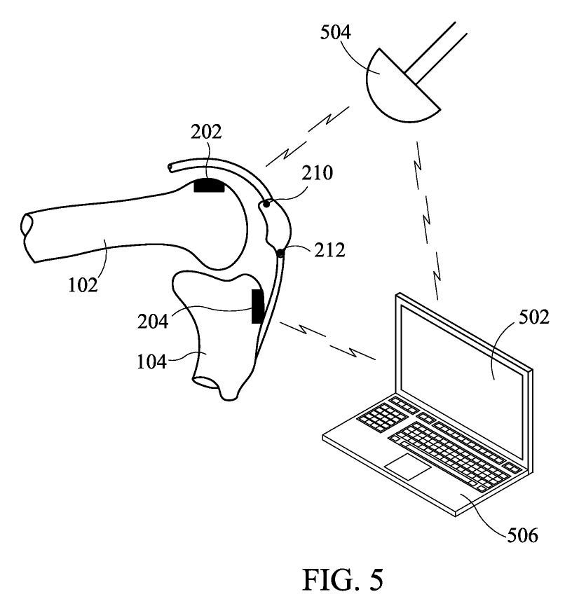

FIG. 5 is a lateral view of the plurality of sensor arrays in communication with a processing unit 506 and a screen 502 for providing information in accordance with an exemplary embodiment. In at least one exemplary embodiment, sensor arrays 202, 204, 206, 208, 210 and 212 are in communication with a computer or computational device having processing unit 506 for processing information from the sensors. For example, processing unit 506 can be a microprocessor, a microcontroller, a digital signal processing chip, a mixed signal analog/digital chip, a logic circuit, a notebook computer, a personal computer to name but a few. Screen 502 is coupled to the computer for displaying sensor array measurement and position information. In a non-limiting example, screen 502 and the computational device are outside of the surgical zone (or sterile box) in an operating room. In one embodiment, processing unit 506 and screen 502 comprises a notebook computer for portability, lower cost, and minimizing footprint in the operating room. The notebook computer will incorporate a user interface for use by the surgeon or medical professionals that allow real time interaction with the sensor position and measurement information. For example, as an aid to the surgeon, the portion of the skeletal structure having sensor arrays placed thereon can be displayed on screen 502 to show alignment, position, and relational positioning in real time as the surgical procedure progresses. Thus, the surgeon has a tool that combines both position and parameter measurement to aid in ensuring correct positioning of an implanted device, that the implanted device parametrics measure within reason, and allowing adjustments to be made and measured thereby allowing a surgeon to subsidize qualitative information with quantitative data.

In at least one exemplary embodiment, element 504 facilitates communication between the sensor arrays and processing unit 506. Element 504 comprises receive and send circuitry and is in communication with processor unit 506 and sensor arrays 202, 204, 206, 208, 210, and 212. Element 504 can be placed in proximity to the sensors to ensure pick up of the signal. For example, component 504 can be incorporated into a lighting system of the operating room where it has a direct and unblocked communication path. Alternatively, the element 504 can be incorporated into the housing for the computational device or screen 502 to provide the sensor information to the processor. Element 504 can be directly connected to sensors 202, 204, 206, 208, 210, and 212 by wires or fiber optics. Similarly, element 504 can be connected to processing unit 506 by wire or fiber-optics. Element 504 can also be wirelessly connected to sensors 202, 204, 206, 208, 210, and 212 and the processor using radio frequency, ultrasonic, infra-red, magnetic or other wireless communication methodology.

As mentioned previously, each sensor array is coupled to a control circuit. The control circuit includes circuitry to convert the data to a form that can be transmitted by wire or wirelessly. For example, the control circuit can have transmitter/receiver circuitry for transmitting data in a known format such as Bluetooth, UWB, or Zigbee. In one embodiment, position and measurement data is taken periodically or by command. The data can be stored in memory. The control circuit can be enabled by a received signal from processing unit 506 to send the information stored in memory. Similarly, the control circuit can be enabled to take position and measurement data by processing unit 506. This enables multiple sensor arrays to be enabled and an orderly process for collecting data, sending data, analyzing processing the information (using processing unit 506), and displaying the data on screen 503 for use by the surgeon or medical team during surgery.

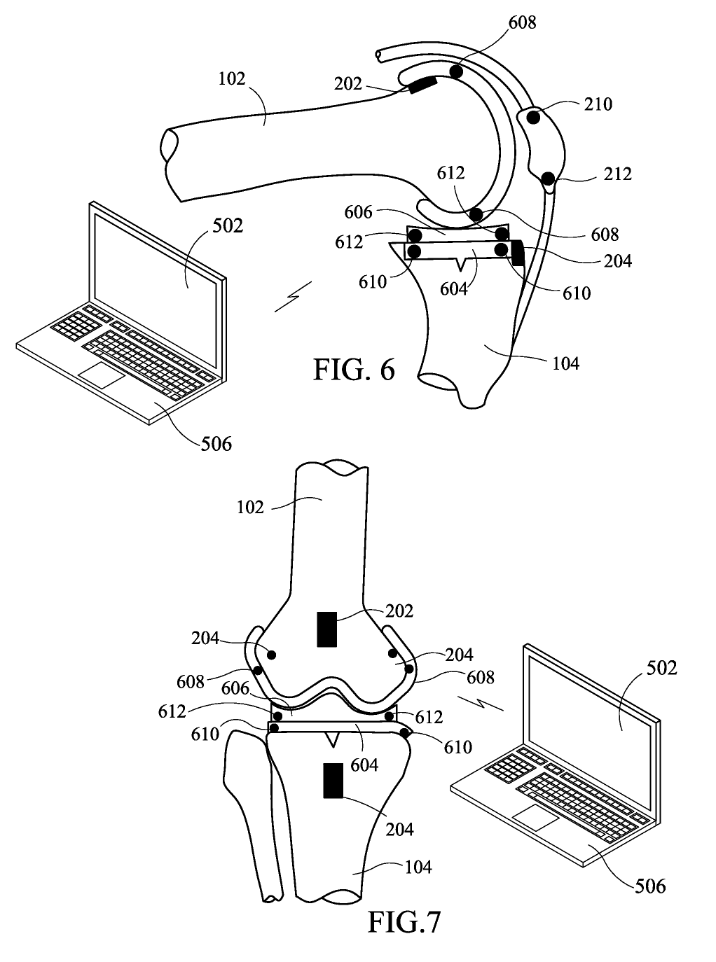

FIG. 6 is a lateral view of the knee illustrating a knee with a joint implant and sensors in accordance with an exemplary embodiment. The knee is used as an example of the system for orthopedic implants to lower cost, reduce stress on the patient, have a small spatial footprint in the operating room, collect data, aid in tuning the device implant for optimal geometry, and reduce short term/long term post-operative rework. The system is adaptable for use in all areas of the skeletal system. More specifically, a single system is disclosed for orthopedic surgery, which can provide alignment, positioning, relational positioning, initial conditions, loading, and balance information over the entire range of motion. Integration into a single system greatly simplifies the procedure and ensures consistency of results because both qualitative (e.g. surgeon) and measured (quantitative) data can be used to assess each step of the procedure. Moreover, the data collected can be used to identify issues before they become problems for the patient and provide information for improving the orthopedic device.

There is a general trend to implement solutions that lower health care operating costs without compromising patient care. One benefit of the system is that it can be easily incorporated into orthopedic surgeries because of low cost. The single system does not require a significant capital expense. For example, the computational device that houses processing unit 506 can be a laptop computer that can be purchased at low cost instead of a fully customized system. Software corresponding to this application would be downloaded to the laptop computer. Element 504 can also be coupled to the laptop computer either wired or wirelessly to support communication if needed. In at least one exemplary embodiment, the system is made as a disposable device. In other words, there is almost no capital expense required by the hospital or clinic to implement the system thereby eliminating typical barriers to adopting new technology. Some of the system components are incorporated in orthopedic implant trials or temporarily attached to the skeletal system, these parts can be disposed of after measurements are made or prior to the final implant device installation. Alternatively, the sensors can be permanently incorporated into the skeletal structure and final implant device for post-operative monitoring and for long term device monitoring.

In a non-limiting example, the implanted device is shown with a trial insert used to measure and tune the knee joint prior to a final insert being installed. The single system comprises the sensor arrays disclosed hereinabove. The single system further comprises femoral implant 602, tibial implant 604, and trial insert 606. Trial insert 606 measuring measures a parameter such as load over a range of motion. In at least one exemplary embodiment, the knee joint is exposed by incision. Alignment of the mechanical axis of the lower leg is achieved as disclosed above with the leg in extension such that the femoral head center, medial tibial spine, and ankle center are aligned in a straight line using the single system to aid the surgeon. Bony cuts are made utilizing the alignment whereby the distal end of femur 102 and the proximal end of tibia 104 are shaped for receiving orthopedic joint implants. Jigs and other orthopedic devices can be used to shape and aid in the bony cuts. The sensors can be attached to the cutting jigs or devices to aid the surgeon in optimizing the depth and angles of their cuts.

In a non-limiting example, a rectangle is formed by the bony cuts. The imaginary rectangle is formed between the cut distal end of femur 102 and the cut proximal end of tibia 104 in extension and in conjunction with the mechanical axis of the lower leg. A predetermined width of the rectangle is the spacing between the planar surface cuts on femur 102 and tibia 104. The predetermined width corresponds to the thickness of the combined orthopedic implant device comprising femoral implant 602, trial insert 606, and tibial implant 604. Trial insert 606 is inserted between the installed femoral implant 602 and tibial implant 604. Trial insert 606 can have a surface comprising the same or similar material as a final insert.

In at least one exemplary embodiment, trial insert 606 comprises load, accelerometer, and other types of sensors. The sensors are in communication with processing unit 506. Sensors can be placed in femoral implant 602, trial insert 606, and tibial implant 604 that work in conjunction with the sensors described hereinabove to define limb alignment, implant-to-implant alignment, and joint kinematics. In general, the sensors of in femoral implant 602, trial insert 606, and tibial implant 604 can measure parameters such as weight, strain, pressure, wear, position, acceleration, temperature, vibration, density, and distance. Trial insert 606 is used to measure the load on either condyle surface of femoral implant 602 while in extension. In a non-limiting example, the screen of the system (not shown) can show the location of the point of contact for both condyle surfaces on trial insert 606 and the load.

Trial insert 606 can indicate that the loading measurement on both condyles is either high, within an acceptable predetermined range, or low. A loading that measures above a predetermined specification can be adjusted using a thinner final insert. Conversely, a loading that measures below a predetermined specification can be adjusted using a thicker final insert. The system can provide an appropriate solution from a look up table (changes in thickness versus measurement to get within a predetermined range). Alternatively, trial insert 606 can be removed and another trial insert of a different thickness can be used to take a measurement such that a loading in the predetermined range is measured. The surgeon can also make a soft tissue adjustment in the case where the tension is too high but close to the predetermined range. As mentioned previously, the system is in communication with processing unit 506 to record measurements during the surgical procedure.

Balance is a comparison of the load measurement of each condyle surface. Balance correction is performed when the measurements exceed a predetermined difference value. Soft tissue balancing is achieved by loosening ligaments on the side that measures a higher loading. The system provides the benefit of allowing the surgeon to read the reduced loading on screen 502 of the system with each soft tissue release until the difference in loading between condyles is within the predetermined difference value. Another factor is that the difference in loading can be due to surface preparation of the bony cuts on either femoral implant 602 or tibial implant 604. The surgeon has the option of removing bone to on either surface underlying the implant to reduce the loading difference. In a further embodiment, trial insert 606 provides position data where each contacts a surface of trial insert 606. Similar to above, the surgeon has the option of altering the surface of the distal end of femur 102 or the proximal end of tibia 104 to move the contact regions in conjunction with the mechanical axis.

As shown, the lower leg is in flexion with tibia 104 at a right angle to femur 102. In general, one or more bony cuts to the distal end of femur 102 are made. In particular, a prepared surface at the distal end of femur 102 is parallel to the prepared surface of tibia 104 in this position. Similar to that described above, an imaginary rectangle is formed by the parallel surfaces of femur 102 and tibia 104 in the ninety-degree flexion position. A predetermined width of the imaginary rectangle is the spacing between the planar surface cuts on femur 102 and tibia 104 in the flexion position (ninety degrees). The predetermined width corresponds to the thickness of the combined orthopedic implant device comprising femoral implant 602, trial insert 606, and tibial implant 604. Ideally, the measured width is similar or equal to the width of the imaginary rectangle in extension. Load measurements are made with the leg in flexion. Adjustments to the load value and the balance between condyles can be made by soft tissue release, and femoral cuts/implant rotation. Once adjusted, tibia 104 can be moved in relation to femur 102 over the range of motion. The loading can be monitored on the screen over the range of motion to show that the absolute loading on the knee is within a predetermined load range and that the difference in loading between the two condyles is within a predetermine differential value. Should an out of range/value condition occur, the surgeon can view on screen 502 of the system the position where it occurs and can take steps to bring it within specification. It should be noted that the surgeon does not have this capability now. Finally, as the leg is rotated through the range of motion a plot of the movement of the contact region of either condyle can be plotted on the screen. The contact region should be within a predetermined area. Movement outside the predetermined area can indicate a misalignment or rotation issue, which the surgeon can correct at this time. The trial insert is removed if the surgeon is satisfied by the measured data. Femoral implant 602, a final insert, and tibial implant 604 are then permanently attached to the knee. In at least one exemplary embodiment, the final insert can have sensors for post-operative monitoring and long term monitoring of the implanted device.

Sensor arrays 210 and 212 on patella 112 can be used to track position and measure a parameter (such as load). Sensor arrays 210 and 212 work with sensor arrays 608 in femoral implant 602. Moving the leg through a range of motion will track patella 112 in relation to femoral implant 602. The system will show patellar movement and loading on the screen. The surgeon can then use soft tissue adjustments and/or a change in the implant rotation positioning to ensure the patella tracks correctly (alignment) and that the loading stays within a predetermined range (over the range of motion). With each correction, the surgeon can view on the screen how the correction affected patellar tracking and loading until satisfactory results are achieved. It should be noted that surgeons do not have this feedback at this time to make adjustments.

FIG. 7 is an anteroposterio view of a knee and sensor arrays in accordance with an exemplary embodiment. The sensor arrays are incorporated for long term monitoring. Femur 102 is shown having sensor arrays 202. Femoral implant 602 is coupled to the distal end of femur 102. Femoral implant 602 includes sensors 608. Tibia 104 is shown having sensor arrays 204. Tibial implant 604 is coupled to the proximal end of tibia 104. Tibial implant 604 includes sensor array 610. An insert 606 is coupled between tibial implant 604 and femoral implant 602. Two condyles of femoral implant 602 ride on a bearing surface of insert 606. Sensor arrays (not shown) underlying the bearing surface of insert 606 can be used to take measurements as disclosed hereinabove. The sensors of the system work in conjunction with processing unit 506 and communication circuitry to provide data that can be used to determine the working status of the implant and to minimize short term and long-term problems after surgery. In at least one exemplary embodiment, the patient can return for outpatient review of the implant. The sensor arrays of the system can be placed in communication with processing unit 506 or another system loaded with enabling software. An analysis of the status of the orthopedic device and patient health can be provided and displayed on screen 502.

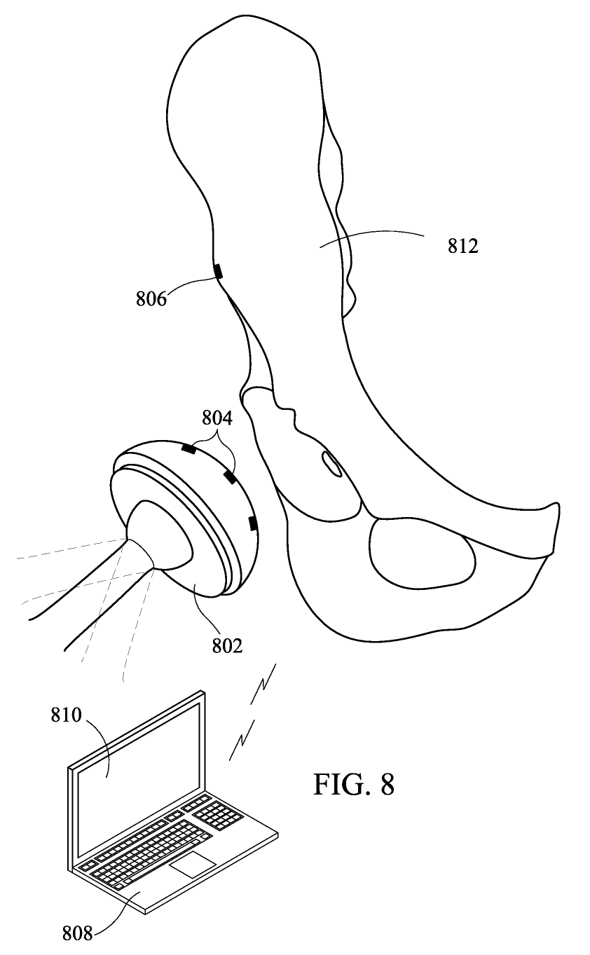

FIG. 8 is an illustration of a system 800 having sensor arrays in accordance with an exemplary embodiment. The system disclosed is a non-limiting example used in the installation of an orthopedic device for a hip replacement. The appropriate kinematics of the hip joint is achieved by implant alignment and refined by increasing or decreasing the hips offset or the limb length. One or more sensor arrays 806 are coupled to the pelvis and one or more sensor arrays are placed in the femur prior to the hip being dislocated. Sensor arrays 806 provide position and measurement data on the existing joint that can be compared later to the implanted joint or during refinement of the implant to inform the surgeon of the hip joint function. It should be noted that the depiction of the hip joint sensor integration can be utilized in other areas of the skeletal system.

The system comprises one or more tools and implanted orthopedic devices incorporating sensor arrays in communication with a processing unit 808. The system measures and displays parameters of the hip joint including load, position, relational positioning, distance, geometry, and other parameters disclosed hereinabove (e.g. knee example). In general, the damaged portions of the hip joint are replaced. Typically, the femoral head of the femur is removed and the acetabulum is shaped. The acetabulum is a partial spherical shaped bony region in the pelvis that receives the femoral head. It cannot be understated that the orthopedic implants have an orientation and geometry similar to the original bone structure. This can only be achieved if the implanted orthopedic devices can be oriented correctly (hip to pelvis) with similar physical geometry and symmetry. Incorrect replacement can lead to hip dislocation, one leg being longer or shorter than the other, instability, and other movement difficulties after implantation.

The acetabulum in the pelvis is shaped with a reaming tool 802 of the system that removes bony material and cartilage in the region. Reaming tool 802 includes sensor arrays 804 that define the varying depths and angles in three planes as the acetabulum is shaped. A trial cup will be inserted that is similar in size to the patient's natural cup to define the starting angles. Sensor arrays 806 in the pelvis define the planes of the pelvis. In at least one exemplary embodiment, sensor arrays 806 comprise accelerometers. Sensor arrays 804 and 806 are in communication with a processing unit 808. As the reamer is installed, sensor arrays 804 will maintain the visual positioning the surgeon wants to achieve. This process an be used in cutting instruments/reamers during knee, shoulder, ankle joint, spine surgery. Processing unit 808 processes information from reaming tool 802 and displays positional and shape information of the material removal process on a screen 810. Once the acetabulum is shaped, the trial cup (socket) is selected to be fitted into the shaped acetabulum.

Typically, an interference fit is used to hold the cup in the acetabulum. A cup is selected that is slightly larger than the opening. Glue can also be used to ensure a secure fit if the surgeon deems it necessary. At this time, the fitting of the cup is difficult because two angles in relation to the pelvis must be contemplated in the insertion process. In at least one exemplary embodiment, an impaction instrument is fitted with sensors similar to reaming tool 802 to enable the surgeon to define cup orientation. For example, accelerometers can be used to monitor position and relative positioning of the impaction instrument. In particular, the accelerometers will allow the orientation in three planes to achieve appropriate anteversion, opening and depth.

The impaction instrument fits into a trial cup and includes a handle that can be rotated to direct a force applied to the end of the handle to a specific region of the cup thereby positioning the cup in the acetabulum. The sensors of the cup impaction instrument are in communication with processing unit 808. The sensors provide positional information of the impaction instrument (and thereby the trial cup) in relation to the pelvis. Screen 810 can indicate when the handle is positioned correctly to drive the cup in at the appropriate angles to seat the acetabular cup fully and define full stability. The surgeon can then use a mallet to drive in the cup. In a non-limiting example, reamer and impaction tool can be part of the same tool.

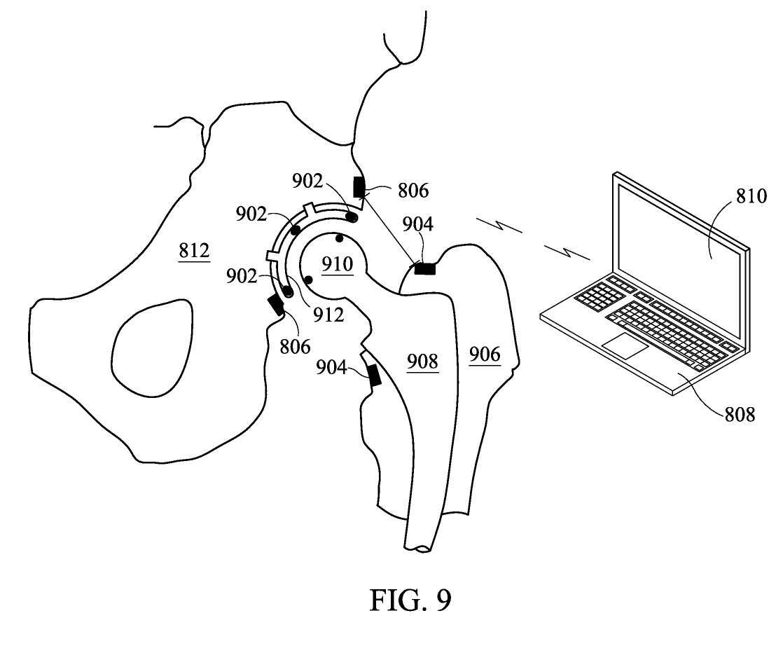

FIG. 9 is an illustration of a hip implant having sensors in accordance with an exemplary embodiment. A proximal end of a femur 906 has been prepared for receiving a femoral implant 908. The femoral implant includes a femoral head 908 that is fitted into a trial cup 912. In at least one exemplary embodiment, sensor arrays 904 are in or attached to femur 906. The femoral head of the implant can also include sensor arrays. In at least one exemplary embodiment, sensor arrays 902 are placed in trial cup 912. Sensors 806, 902, and 904 are in communication with processing unit 808 for providing location and distance information that is displayed on screen 810. In particular, the system can make a distance measurement that ensures that femoral implant 908 results in an appropriate leg length. More specifically, a distance measured between sensors 806 and sensors 904 corresponds to a length measured prior to installing femoral implant 908. The distance of installed femoral implant 908 should be similar to that of the prior spacing. An incorrect distance can result in a different leg length than the person had originally which is very noticeable and source of complaint by hip replacement patients. The joint offset can also be measured and displayed on screen 810 using the sensor arrays to display the working hip joint in three-dimensional space. The surgeon can make further adjustments to prevent rework or potential problems at this time based on measurements of the actual implanted joint thereby ensuring the best fit possible.

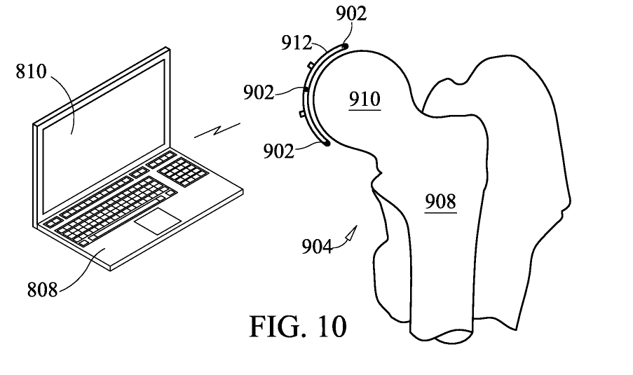

FIG. 10 is an illustration of a hip implant having load sensors 902 in accordance with an exemplary embodiment. System 800 measures appropriate implant and implant articulation. In general, femoral head 910 of femoral implant 908 is made of metal that articulates with a polymer or another metal that forms a bearing surface in the acetabulum. If the alignment of the prostheses is not optimal, the implants can impinge on each other leading to edge loading, early implant wear, and dislocation.

As mentioned above, trial cup 912 includes load sensors 902. Load sensors 902 are positioned in different regions of the trial cup and are in communication with processing unit 808. Once inserted, measurements of the loading in different areas of trial cup 912 can be made and displayed on screen 810. The loading measured by sensors 902 should be within a predetermined range. The cup may not be fully seated if the measurement is outside the range.

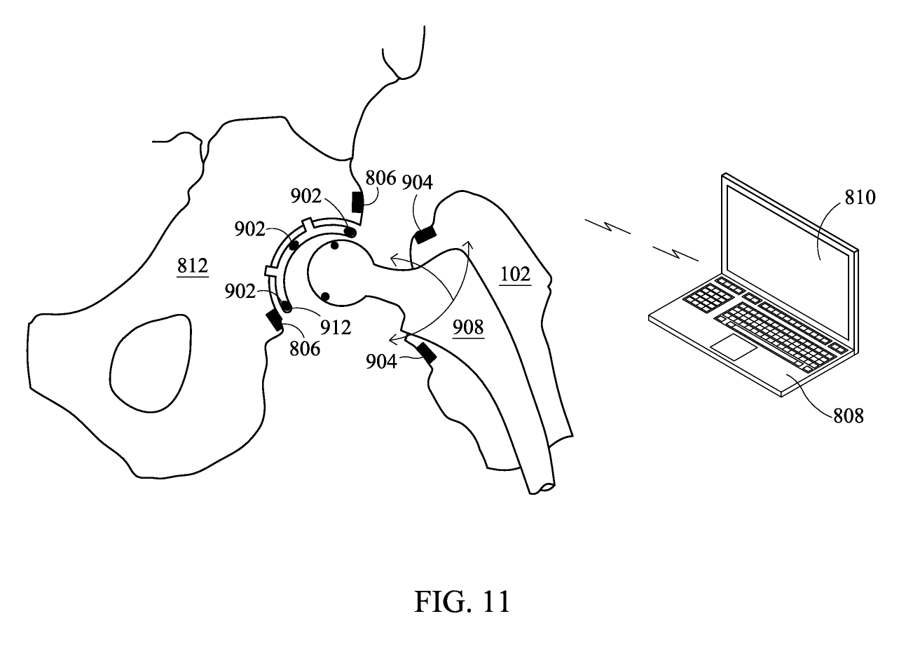

FIG. 11 is an illustration of moving the hip implant to measure load and position through a range of motion in accordance with an exemplary embodiment. Sensors 806, 902, and 904 provide position and load information to processing unit 808. The position of the pelvis and hip in relation to each other can be displayed on screen 810. Load measurements are taken by sensors 902 on cup 912 as the hip is moved over the entire range of motion. The surgeon can use the real time measurements to balance the loading over the range of motion through ligament tensioning and implant positioning. In general, the femoral head 910 defines that that cup 912 is fully seated and femoral head 910 is equally loading the geometry of cup 912 as the sensors define the position of the joint. This will allow the surgeon to rotate the insert, reposition the cup or femoral implant to achieve optimal implant to implant articulation through all degrees of motion and define any aspects of instability or overload.

Fine tuning of the implant can be made utilizing the alignment and load measurements in three dimensions. The impaction instrument can be used to make fine adjustments in placement of cup 912 by positioning the handle and applying a force to move the cup within the acetabulum. The surgeon can be directed to apply the force in an appropriate direction by processing unit 808 to position cup 912 using an analysis of the data that is viewed on screen 810 (e.g. current position versus ideal position). Thus, the system can provide both alignment, positional, relational positioning, loading and other measured parameters that aids the surgeon in the installation of cup 912 and femoral implant 908 such that it is fitted very accurately thereby reducing post-operative complications for a patient.

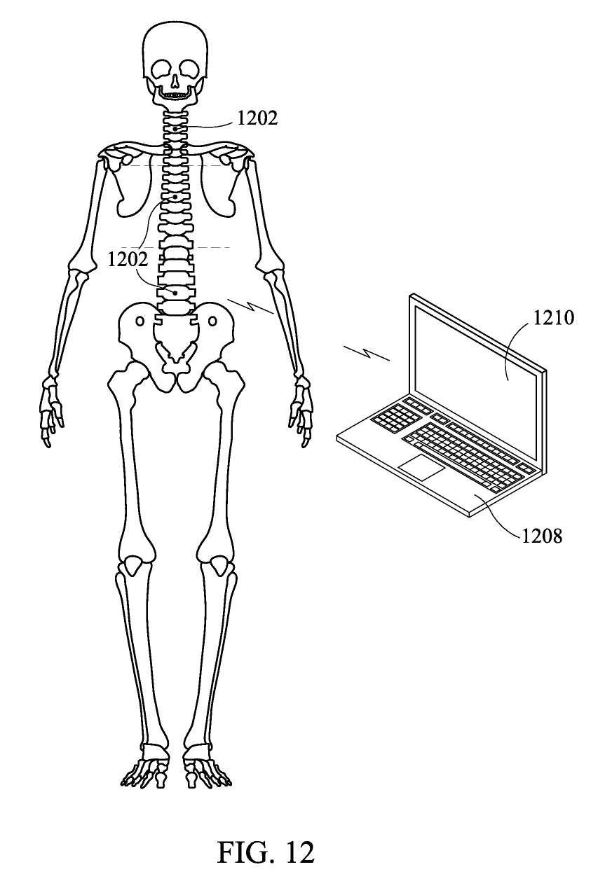

FIG. 12 is an illustration of a spinal column and sensors in accordance with an exemplary embodiment. The human spine comprises a cervical, thoracic, and lumbar regions respectively corresponding to C1-07, T1-T12, and L1-L5. A healthy spinal column has a mechanical axis in an upright position that distributes loading that minimizes stress on each vertebrae. An example of a spinal deformity that can require correction is scoliosis, which is a curving of the spine. In general, spinal deformities can often be corrected using devices that place the spine or help the spine be in the most ideal mechanical situation. In any spinal correction, the position of the spine and each element of the spine needs to be in alignment and dimensionally correct (in all three dimensions). Thus, in spine surgery, alignment and stability are critical and often difficult to achieve. It is important for the surgeon to obtain data as he/she corrects the spinal deformity in 3 planes. It is also helpful to identify the increasing and decreasing loads across spinal segments as this is performed.

A system includes more than one sensor arrays 1202. In at least one exemplary embodiment, at least one sensor is placed on or in the cervical, thoracic, and lumbar regions of the spinal column. In a non-limiting example, sensor arrays 1202 include accelerometers or other position sensing devices such as fiber—optics, RF/EM/US sensors that detect position in all three dimensions. In particular, the placement of sensor arrays 1202 on a vertebrae is done in a manner where the three-dimensional position data reflects the position of the vertebrae of the spinal column. Sensor arrays 1202 are in communication with computational unit 1208 for providing three dimensional positioning information on screen 1210 of the vertebrae and the regions of the spine. It should be noted that sensors 1202 provides positional information in relation to each sensor and can provide data corresponding to the rotation of a vertebrae within a region of the spine or from region to region. As shown, screen 1210 would display (in varying views) that the vertebrae of the spinal column are aligned along the preferred mechanical axis or an axis corresponding to each spinal region in three dimensions and that each vertebrae are not rotated in the mechanical axis.

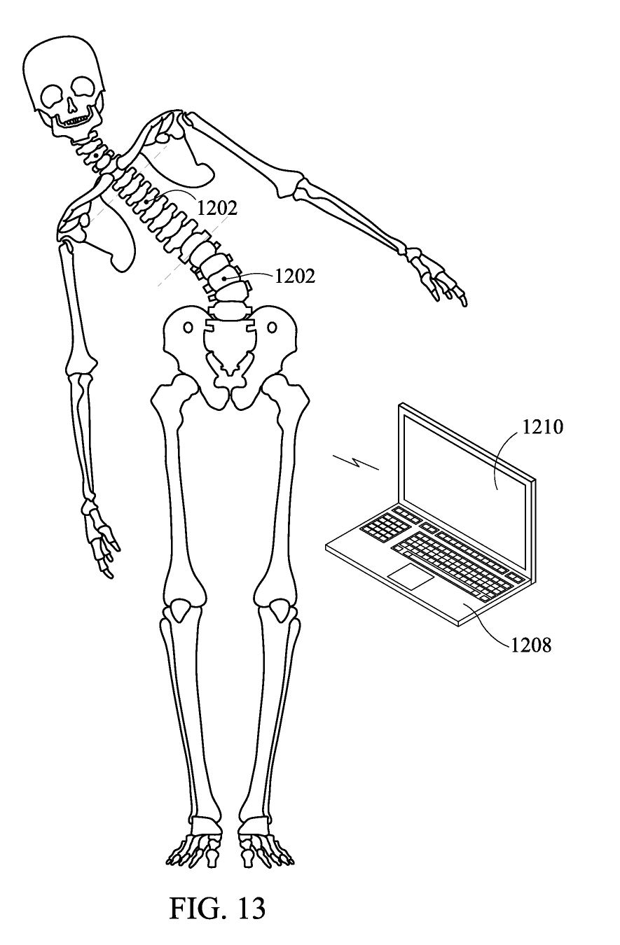

FIG. 13 is an illustration of a spinal column and sensors providing positional information in accordance with an exemplary embodiment. As illustrated, sensor arrays 1202 are placed in predetermined locations of the spinal column. Sensor arrays 1202 in communication with computational unit 1208 indicate curvature of the spine in more than one spinal region on screen 1210. The surgeon can view the definition of the pre-surgical alignment in all three planes on screen 1210. In at least one exemplary embodiment, the surgeon will be able to rotate the image on the screen to see spine alignment from different perspectives.

The surgeon will use the system during surgery to further define the achievement of the overall spinal correction angle, and define that the cervical sacral angles are centralized. The surgeon adds bracing, adjusts tensioning, or utilizes other techniques known to one skilled in the art to maintain the spine in position. Adjusting one area of the spine may disrupt or change positions in other areas of the spinal column. The system provides information to these changes and allows the surgeon to compensate while the surgery takes place.

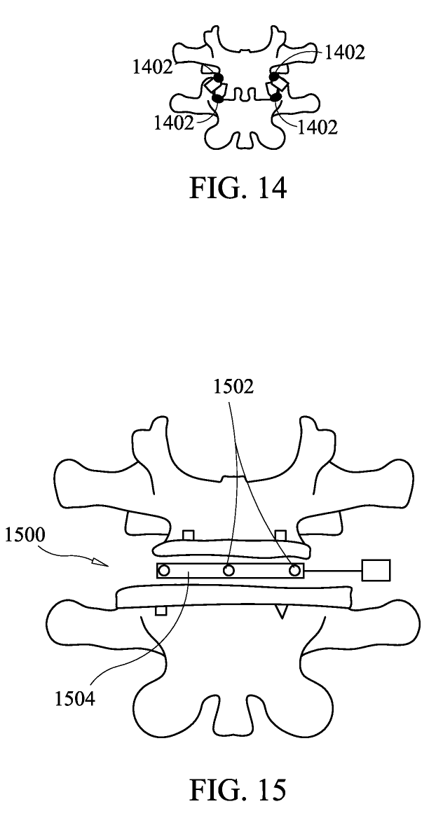

FIG. 14 is an illustration of vertebrae having one or more sensor arrays 1402 in accordance with an exemplary embodiment. The illustration shows sensors 1402 monitoring adjacent vertebrae. Sensor arrays 1402 are placed in or on the vertebrae such that the force or loading between the two vertebrae can be measured. In at least one exemplary embodiment, the loading can be measured circumferentially to determine if unequal forces are applied to different areas of the vertebrae. Position measurements using sensors 1402 can show whether adjacent major surfaces of the vertebrae are parallel to one another and perpendicular to the mechanical axis. Similarly, position data from sensors 1402 can indicate if the vertebrae are rotated from an ideal alignment. Although load is being measured in the example, sensors 1402 can measure on or more of at least load, weight, strain, pressure, wear, position, acceleration, temperature, vibration, density, and distance to name a few. Thus, substantial benefit can be provided by the system that combines position, alignment, relational positioning, with measurement of one or more parameters in real time to aid in correct installation of an orthopedic device. It also allows sensing of changes in vascular flow, neural element function that would aid in detecting changes at the operative site.

FIG. 15 is an illustration of a spinal implant and cage in accordance with an exemplary embodiment. In at least one exemplary embodiment, sensor arrays 1502 can be used to define appropriate balance of the spinal implant during surgery such as a disc implant or fusion cage. In a non-limiting example, sensor arrays 1502 are placed in a trial insert for measuring position and load. The load sensors can define the increased or decreased loads seen above an instrumented spinal segment. This will allow motion preserving implants to be utilized without severely affecting the mechanics of adjacent joint segments. These sensors can be disposed of after surgery, or left in to define post operative angles and loads.

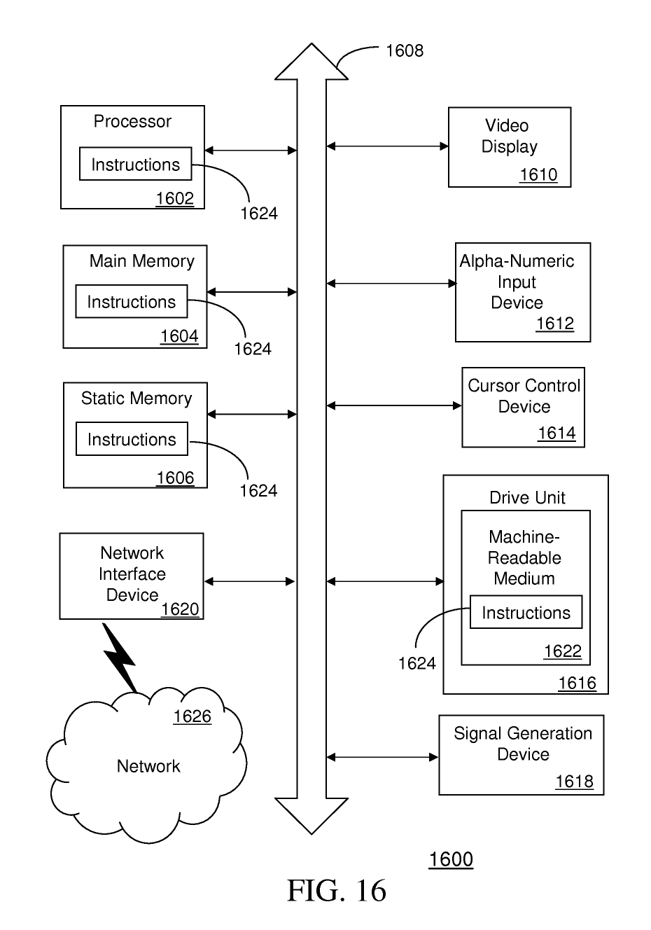

FIG. 16 depicts an exemplary diagrammatic representation of a machine in the form of a computer system 1600 within which a set of instructions, when executed, may cause the machine to perform any one or more of the methodologies discussed above. In some embodiments, the machine operates as a standalone device. In some embodiments, the machine may be connected (e.g., using a network) to other machines. In a networked deployment, the machine may operate in the capacity of a server or a client user machine in server-client user network environment, or as a peer machine in a peer-to-peer (or distributed) network environment.

The machine may comprise a server computer, a client user computer, a personal computer (PC), a tablet PC, a laptop computer, a desktop computer, a control system, a network router, switch or bridge, or any machine capable of executing a set of instructions (sequential or otherwise) that specify actions to be taken by that machine. It will be understood that a device of the present disclosure includes broadly any electronic device that provides voice, video or data communication. Further, while a single machine is illustrated, the term “machine” shall also be taken to include any collection of machines that individually or jointly execute a set (or multiple sets) of instructions to perform any one or more of the methodologies discussed herein.

The computer system 1600 may include a processor 1602 (e.g., a central processing unit (CPU), a graphics processing unit (GPU, or both), a main memory 1604 and a static memory 1606, which communicate with each other via a bus 1608. The computer system 1600 may further include a video display unit 1610 (e.g., a liquid crystal display (LCD), a flat panel, a solid state display, or a cathode ray tube (CRT)). The computer system 1600 may include an input device 1612 (e.g., a keyboard), a cursor control device 1614 (e.g., a mouse), a disk drive unit 1616, a signal generation device 1618 (e.g., a speaker or remote control) and a network interface device 1620.

The disk drive unit 1616 may include a machine-readable medium 1622 on which is stored one or more sets of instructions (e.g., software 1624) embodying any one or more of the methodologies or functions described herein, including those methods illustrated above. The instructions 1624 may also reside, completely or at least partially, within the main memory 1604, the static memory 1606, and/or within the processor 1602 during execution thereof by the computer system 1600. The main memory 1604 and the processor 1602 also may constitute machine-readable media.

Dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the present disclosure, the methods described herein are intended for operation as software programs running on a computer processor. Furthermore, software implementations can include, but not limited to, distributed processing or component/object distributed processing, parallel processing, or virtual machine processing can also be constructed to implement the methods described herein.

The present disclosure contemplates a machine readable medium containing instructions 1624, or that which receives and executes instructions 1624 from a propagated signal so that a device connected to a network environment 1626 can send or receive voice, video or data, and to communicate over the network 1626 using the instructions 1624. The instructions 1624 may further be transmitted or received over a network 1626 via the network interface device 1620.

While the machine-readable medium 1622 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present disclosure.

The term “machine-readable medium” shall accordingly be taken to include, but not be limited to: solid-state memories such as a memory card or other package that houses one or more read-only (non-volatile) memories, random access memories, or other re-writable (volatile) memories; magneto-optical or optical medium such as a disk or tape; and carrier wave signals such as a signal embodying computer instructions in a transmission medium; and/or a digital file attachment to e-mail or other self-contained information archive or set of archives is considered a distribution medium equivalent to a tangible storage medium. Accordingly, the disclosure is considered to include any one or more of a machine-readable medium or a distribution medium, as listed herein and including art-recognized equivalents and successor media, in which the software implementations herein are stored.

Although the present specification describes components and functions implemented in the embodiments with reference to particular standards and protocols, the disclosure is not limited to such standards and protocols. Each of the standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same functions are considered equivalents.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. A system comprising:
one or more location sensors coupled to a skeletal system to measure position, relational positioning, and alignment;
one or more measurement sensors coupled to a skeletal system for measuring parameters of the skeletal system; and
a processing unit that receives both positioning information and at least one measurement parameter to generate a measurement where one or more bones of the skeletal system are in a predetermined alignment.

2. The system of claim 1 where the one or more location sensors are coupled to bones of a skeletal system to provide data in three dimensions and where a position of a sensored bone is displayed on a screen.

3. The system of claim 1 where the one or more locations sensors provide information of a pre-operative skeletal system range of motion and where the processing unit compares the pre-operative range of motion to a range of motion of an implanted device.

4. The system of claim 1 where the one or more locations sensors are coupled to the lower leg to provide positional information on the femur and tibia to the processing unit, where the processing unit in conjunction with position data provided by the one or more location sensors determines when the mechanical axis of the lower leg in extension is aligned, and where the one or more location sensors can provide relational positioning data of a tibia in relation to a femur of the lower leg.

5. The system of claim 4 further including a trial insert for an implanted knee joint having one or more measurement sensors where the trial insert is in communication with the processing unit and where the trial insert in conjunction with the more than one location sensors displays provides measurement and position information to the processing unit.

6. The system of claim 5 where the trial insert includes at least one positional sensor in communication with the processing unit.

7. The system of claim 6 where the trial insert is inserted between the distal end of the femur and the proximal end of the tibia, where the trial insert measures at least load for a predetermined trial insert thickness such that an appropriate final insert thickness from the measurement, and where the loading is within a predetermined range with the final insert in place.

8. The system of claim 7 where the system measures balance in each compartment of a knee with the trial insert, where the system indicates that the lower leg the mechanical axis is correctly aligned when the measurement is taken, and providing information for soft tissue balancing to balance the knee.

9. The system of claim 5 further including a final insert for an implanted knee joint having one or more measurement sensors and one or more location sensors for long term monitoring of an implanted knee joint.

10. The system of claim 1 where at least one location sensor is coupled to each of a cervical region, thoracic region, and a lumbar region of a spinal column to provide positional information to the processing unit, where the processing unit in conjunction with position data provided by the one or more location sensors determines a mechanical axis of the spinal column in three dimensions, and where corrections to a spinal column are reported in positional relation to the mechanical axis.

11. The system of claim 10 where the one or more measurement sensors are coupled between vertebrae, where a correction is applied to the spinal column, and where the one or more positional sensors in conjunction with the computational unit indicate a degree of positional correction achieved and the loading on the sensored vertebrae before and after correction.

12. The system of claim 10 further including a trial insert placed between vertebrae where the trial insert has one or more measurement sensors, where the trial insert is in communication with the processing unit, where the trial insert in conjunction with the more than one location sensors provides measurement and spinal column alignment information to the processing unit.

13. The system of claim 12 where the trial insert includes one or more position sensors, where the trial insert measures load at more than one point between the vertebrae to determine if an imbalance exists and providing information to correct the imbalance such that the verterbrae are aligned to the mechanical axis and loads are distributed evenly on contacting surfaces of the vertebrae.

14. The system of claim 1 where the one or more location sensors includes at least one location sensor coupled to a pelvis and at least one location sensor on a reaming tool where positional information provided by the one or more location sensors defines the varying depths and angles in three planes as the reaming tool shapes the acetabulum.

15. The system of claim 14 where at least one or more location sensors are coupled to the femur such that a distance between the acetabulum and femur can be measured before and after implanting a hip joint to define leg offset and joint offset and providing information to correct length and offset.

16. The system of claim 14 where at least one or more location sensors are coupled to an impaction instrument, where the sensors of the impaction instrument are in communication with the processing unit, where the processing unit calculates and illustrates on the screen an appropriate alignment of the impaction instrument in three dimensions to apply force to seat the cup in the acetabulum.

17. The system of claim 16 where at least one or more of the measurement sensors are coupled to a trial cup, where the measurement sensors in the trial cup are load sensors, where the load sensors are in communication with the processing unit, where the loading of the hip joint can be measured through a range of motion in conjunction with the position sensors, and where proper seating, implant stability, and balance can be determined and corrective measures taken if outside a predetermined range.

18. A method of generating orthopedic information comprising the steps of:
using a trial insert in a joint of the skeletal system where the trial insert includes one or more measurement sensors to measure a parameter of the joint;
coupling one or more position sensors to the skeletal system where the measurement sensors and the position sensors are in communication with a processing unit;
communicating position data of the joint with each measurement to the processing unit; and
measuring joint stability and implanted device alignment using the measurements from the measurement sensors and position data.

19. The method of claim 18 further including the steps:
identifying a mechanical axis of the joint using the position sensors such that the implanted device is aligned to the mechanical axis;
measuring loading on the joint; and
comparing the measured loading on the joint to a predetermined range where the predetermined range is based in part on previous measurements from the database.

20. The method of claim 18 further including the steps of;
placing one or more measurement sensors in the implanted device;
measuring parameters associated with joint misalignment, wear and infection; and
communicating the measured data to the database such that the database comprises device implantation measurement and skeletal position data and long-term implant device measurement where the data in the database in part generates predetermined ranges for device installation.

21. A method of using a position and measurement system comprising:
measuring one or more parameters of a skeletal system including a position, relational positioning, or alignment corresponding to the skeletal system;
installing an orthopedic device using quantative measurements of the position and measurement system; and
disposing of a portion of the system after the surgery has been completed.

22. The method of claim 21 further including the steps of:
inserting a trial insert having one or more sensor arrays to measure parameters of the skeletal system prior to installing a final orthopedic device; and
disposing of the trial insert.

23. The method of claim 21 further including the steps of:
attaching temporarily one or more location sensor arrays to the skeletal system; and
disposing of at least one of the location sensor arrays.

24. The method of claim 21 further including the steps of:
attaching temporarily one or more location sensor arrays to components of the orthopedic device; and
disposing of at least one of the location sensor arrays.

25. The method of claim 21 further including the steps of:
attaching temporarily one or more measurement sensor arrays to components of the orthopedic device; and
disposing of at least one of the measurement sensor arrays.
